(19) United States
(12) Patent Application Publication
(10) Pub. No.: US 2016/0331603 A1
(43) Pub. Date: Nov. 17, 2016

(54) ARTICLE FOR ABSORBING A PHYSIOLOGICAL LIQUID, SUCH AS A DRESSING

(71) Applicants: COMMISSARIAT A L'ENERGIE ATOMIQUE ET AUX ENERGIES ALTERNATIVES, Paris (FR); URGO RECHERCHE INNOVATION ET DEVELOPPEMENT, Chenove (FR)

(72) Inventors: Frédéric REVOL- CAVALIER, SEYSSINS (FR); Michel LAMOISE, BESSEY LES CITEAUX (FR); Mouna MESSAOUD, GRENOBLE (FR); Jean-Marc PERNOT, DIJON (FR)

(73) Assignees: COMMISSARIAT A L'ENERGIE ATOMIQUE ET AUX ENERGIES ALTERNATIVES, Paris (FR); URGO RECHERCHE INNOVATION ET DEVELOPPEMENT, Chenove (FR)

(21) Appl. No.: 15/106,599

(22) PCT Filed: Dec. 17, 2014
(86) PCT No.: PCT/IB2014/067009

§ 371 Date: Jun. 20, 2016

(30) Foreign Application Priority Data
Dec. 19, 2013 (FR) 1362987

(51) Int. Cl. A61F 13/537; A61F 13/02

(52) U.S. Cl. CPC A61F 13/53717; A61F 13/022; A61F 2013/53765

(57) ABSTRACT

An article having physiological-fluid absorption properties, including a liquid collection zone and a transfer structure, the latter including absorbent fibers and being designed to transport the liquid from said collection zone toward at least one retention and/or evaporation zone, said transfer structure having a cross section that increases in the direction of said retention and/or evaporation zone, over at least part of the path separating said collection zone from said retention and/or evaporation zone.

The present invention relates to articles intended to come into contact with physiological liquids, particularly liquids secreted by the skin, a wound and/or the mucous membranes in order to manage the propagation, retention and/or evaporation of these liquids and, more particularly although not exclusively, to dressings to be applied to a wound.

The management of the propagation of liquids and retention thereof are complex problems to resolve for which the articles proposed in the field of hygiene products and dressings do not to date provide entirely satisfactory solutions.

These articles have indeed to meet a set of specifications which contains antagonistic requirements.

A first requirement is that the liquids be removed as quickly as possible to as far away as possible to prevent any maceration or irritation of the skin, of the wound or of the mucous membranes caused by the buildup of such liquids. Not only must the liquids not be allowed to accumulate, but it is also preferable to avoid their lateral migration from the site of secretion, so that they do not increase the extent of the moistened zone, this being for the purposes of guaranteeing better hygiene and contributing to the comfort of the user.

This is particularly important in the use of a dressing. In that case, it is of primordial importance to prevent the skin situated around the borders of the lesion, referred to as the perilesional skin, which is very fragile, from becoming moist, because that could damage it, for example encouraging infection and/or irritation.

Thus, it is desirable for the dressing to rapidly remove bodily liquids secreted by the wound away from the site at which they were secreted. Such rapid removal ensures better hygiene and better conditions for healing.

A second requirement is for these liquids to be retained and prevented from returning to the skin, the wound or the mucous membranes. Significant retention makes it possible to increase the length of time for which the article is used. In the case of a dressing, this length of use is of particular importance because it makes it possible to reduce the risk of impairing the wound healing process by changing the dressing less frequently.

Finally, this removal and retention need to work hand in hand for as long as possible regardless of the capacity of the retention zone and in particular of the level of liquid absorbed into the retention zone.

It is therefore desirable to have available articles that make it possible to manage the propagation and retention of liquids, particularly liquids secreted by a wound, the skin or the mucous membranes, by removing these liquids from their point of secretion as quickly as possible in order to prevent them from accumulating and to increase the duration of use of the article.

The use, in the field of dressings, of liquid transfer strips for removing or distributing these liquids in order to evaporate and/or retain them has long been known.

Application EP 541391 proposes incorporating between the absorbent layer and the support a layer that encourages the spreading of the liquids in order to optimize the evaporation thereof. However, that solution is far from optimal because when the spreading layer is saturated with water, evaporation becomes less effective and, above all, the liquids have a tendency to drop back down into the parts of the absorbent layer that are not yet in contact with the liquids. This once again presents the risks associated with the accumulation of liquids at the wound and in the region of the perilesional skin, such as for example, maceration and the possibilities of infection. It also reduces the absorption capacity of the absorbent layer, or even causes it to deform, following the creation of liquid content gradients within it.

Application US 2009 145703 A1 discloses a dressing capable of transferring the exudate absorbed by an absorbent layer positioned under the compression strip to an external reservoir. The transfer of some of the exudate is performed using draining strips which are made of hydrophilic nonwoven based on absorbent fibers.

However, no work has yet been done on optimizing the rate at which the liquids in such transfer strips migrate or in how to maintain this rate of migration over time when the strip becomes completely moist and/or as the retention reservoir becomes full.

In order to optimize liquid management it would therefore be desirable to have available articles which comprise transfer structures that have a rate of migration of liquids that is optimized for draining these liquids from a first point toward a second point with a view to retaining and/or evaporating them.

The invention seeks to create such transfer structures and articles, particularly dressings, that meet the abovementioned objectives.

The invention also seeks to improve hygiene articles other than dressings, such as disposable nappies or feminine sanitary products, by offering a high rate of absorption of bodily leakages, without detracting from user comfort.

One object of the invention, according to a first aspect thereof, is an article having physiological-fluid absorption properties, comprising a liquid collection zone and a transfer structure, the latter comprising absorbent fibers and being designed to transport the liquid from said collection zone toward at least one retention and/or evaporation zone, said transfer structure having a cross section that increases in the direction of said retention and/or evaporation zone, over at least part of the path separating said collection zone from said retention and/or evaporation zone.

The increase in the cross section of the transfer structure is preferably obtained by increasing its width, better by a continuous, notably linear, increase in its width over at least part of said path. The ratio (Iₘₐₓ/Iₘᵢₙ) of the maximum width (Iₘₐₓ) to the minimum width (Iₘᵢₙ) of the transfer structure is preferably greater than or equal to 1.5, preferably 5, or even 10.

The increase in cross section of the transfer structure makes it possible to increase the rate at which liquid is transferred toward the retention and/or evaporation zone.

The invention makes it possible, when the article is a dressing, to optimize the transfer of sonic of the exudate absorbed by a dressing toward the periphery thereof so as to relieve the dressing of its excess exudate.

The transfer structure is preferably arranged at the surface of a collection zone, such as an absorbent layer facing the wound.

The transfer structure allows the liquid to be spread out in order to encourage it to evaporate, and thus prevent the liquid from returning toward the collection zone and the wound. The transfer structure also allows the liquid to be conveyed toward at least an absorbent part forming a retention zone where the liquid may accumulate.

The transfer structure may comprise at least two distinct elements for transporting the liquid toward the retention and/or evaporation zone. In one embodiment of the invention, these elements are, for example, arranged with an angular offset about a center of the article, preferably being evenly angularly distributed. In another embodiment of the invention, the elements extend along substantially mutually parallel axes. “Substantially parallel” encompasses all instances in which the angle between the axes of two elements is not great enough for these axes or the continuations thereof to intersect within the article. The elements therefore preferably form two groups of elements that interpenetrate, notably in alternation, above the collection zone.

The fact that the transfer structure comprises several elements notably allows the elements to drain the liquid in different directions, for example toward at least two respective different retention and/or evaporation zones.

The transfer structure may then conduct the liquid toward one or more peripheral retention and/or evaporation zones, thereby making it possible to be adapted to suit highly exuding wounds, while at the same time maintaining a dressing that is thin and conformable without excessively impairing the retention and/or evaporation properties thereof

Still with a view to optimizing the conformability of the article, the transfer structure may advantageously be non-planar, notably at a receiving absorbent part which defines the collection zone or at the part of the transfer structure that defines the collection zone in the absence of a receiving absorbent part fluidically coupled to the transfer structure. The collection zone of the article may thus be defined by a portion of the transfer structure directly in contact with the source of exudate, in particular directly in contact with the wound.

The elements of the transfer structure may be produced as a single piece by being cut from a material in sheet form, notably a paper or a nonwoven, containing absorbent fibers; the elements are then joined together for example at the collection zone and/or at the retention and/or evaporation zone.

In order to increase the absorption capacity of the article, the elements of the transfer structure may be at least partially superposed, preferably with a liquid-impermeable barrier between two superposed elements, in the region of superposition. This barrier prevents exudates from being transferred between elements. The barrier may be formed from a layer of a material impermeable to liquid, for example a film that is impermeable to liquid but permeable to water vapor.

The invention may thus, if that is called for, make it possible to minimize risks of leakage associated with a build-up of liquid in a particular zone of the article and the risks, in the case of an adhesive dressing, of the dressing becoming detached under the effect of gravity.

The ratio of surface area between any receiving absorbent part there might be and the transfer structure is for example comprised between 50%-50% and 66%-33%, better, 75%-25%.

That may make e t possible to reduce the risk of liquid returning toward the collection zone.

Where there are peripheral retention and/or evaporation zones, limited contact between the transfer structure and a peripheral retention and/or evaporation zone may also limit the return of liquid toward the collection zone.

The article is preferably packaged in the sterile state, notably when it is a dressing.

The article according to the invention may, in one exemplary embodiment of the invention, take the form of a dressing comprising:
a support that is impermeable to water but permeable to water vapor,
a receiving absorbent part in contact with the wound and defining the collection zone, and
a transfer structure according to the invention, which has a cross section that increases with increasing distance away from the receiving absorbent part.

Receiving Absorbent Part and Absorbent Part Forming a Retention and/or Evaporation Zone

The receiving absorbent: part is also termed an “absorbent layer” and the absorbent part forming a retention and/or evaporation zone is also termed a “reservoir-forming layer”. The term “layer” is to be understood as encompassing a monolayer arrangement or several assembled sublayers.

The receiving absorbent part and the absorbent part forming a retention and/or evaporation zone each preferably have a water absorption capacity greater than or equal to 500 g/m², better still greater than or equal to 800 g/m². They may contain or consist of any material capable of retaining liquids, such as for example the materials used in the field of hygiene and dressings.

The absorbent part forming a retention and/or evaporation zone may be defined by a material having a liquid absorption capacity greater than that of the transfer structure. The same is true of the receiving absorbent part. Thus, the water absorption capacity, in g/m², of the transfer structure is preferably less than that of the receiving absorbent part and than that of the absorbent part forming a retention and/or evaporation zone.

By way of example, mention may be made of absorbent foams and preferably hydrophilic polyurethane foams, all materials based on superabsorbent polymer (SAP), such as, for example, absorbent nonwovens incorporating particles of SAP, commonly used in the field of hygiene, absorbent textiles such as, for example, nonwovens based on viscose, rayon or cellulose, such as, for example, a wadding or hydrogels.

Within the context of the present invention it is preferable to use by way of receiving absorbent part as cellular material, for example a hydrophilic polyurethane foam, after the manner for example of the one marketed under the trade name MCF.03 by the company Advanced Medical Solution.

For the absorbent part forming a retention and/or evaporation zone it is preferable to use a material containing a superabsorbent polymer SAP.

The use of nonwovens obtained by the dry route method of manufacture known as the “airlaid” method, which contain particles of SAP and, in particular, between 20 and 60 wt % of SAP with respect to the total weight of the nonwoven is preferable. Such nonwovens are, for example, marketed by the company EAM Corporation under the reference Novathin®.

According to one preferred embodiment of the invention, use is made, for creating the absorbent part forming a retention and/or evaporation zone, of a nonwoven based on particles of superabsorbent polymers and cellulose fibers without the incorporation of thermal binder or latex materials, and which is covered on each of its faces with a cellulose gauze.

According to an alternative form, a material consisting of two cellulose gauzes between which particles of superabsorbent polymers, alone or in combination with binders, are incorporated is used by way of SAP-based material.

According to another alternative form, a material based on SAP fibers alone or in combination with non-absorbent fibers is used. For preference, this material takes the form of a nonwoven.

For preference, the receiving absorbent part is formed of an absorbent layer covered by a wound-contact interface layer, which avoids impairing the wound healing process when the dressing is removed. This interface layer is preferably covered by a temporary protective film which is removed prior to use.

Dressings known by the name of “wound-contact layer interface dressings” such as, for example, the products marketed by the companies Laboratories URGO and MOLNLYCKE HEALTH CARE under the trade names URGOTUL® and MEPITEL® respectively may be used for such a layer.

It is also possible to use polymer-based perforated layers of hydrophobic or hydrophilic formulation but which are non-absorbent or not very absorbent. These formulations may be adherent or nonadherent.

It is preferable to use microadherent or nonadherent formulations. Such formulations are well known to those skilled in the art and are made for example based on silicone gel(s), pressure-sensitive silicone adhesive(s) or compositions containing a block polymer elastomer of the poly(styrene-olefin-styrene) type, a plasticizer such as a mineral oil and a small quantity of hydrocolloid(s) in order to create a moist environment that encourages the healing process without making the composition absorbent in order to avoid blocking the holes. Such microadherent formulations are used for example in the dressings marketed by the company Laboratoires URGO under the trade names URGOCLEAN® and URGOTUL ABSORB®.

According to possible alternative forms, these wound-contact layer interface dressings and these perforated layers of hydrophobic or hydrophilic formulation are combined with permeable materials, particularly non-absorbent nonwovens, which are incorporated between the absorbent layer and the additional layers.

Transfer Structure

The transfer structure is also referred to as the “distribution layer”.

Its role is not to retain the liquids but to allow them to spread.

The transfer structure is preferably less absorbent on the one hand than the receiving absorbent part and on the other hand than the absorbent part forming a retention and/or evaporation zone. The transfer structure preferably has a maximum absorption value of less than 500 g/m².

For preference, its thickness is comprised between 50 and 1000 μm, better still between 300 and 500 μm.

The longest dimension of the transfer structure is, for example, greater than or equal to 15 cm, or even 30 cm. This longest dimension corresponds to the length of the transfer strip.

For preference, the transfer structure comprises or is made of a layer of a hydrophilic material.

For preference, in order to create the transfer structure, fibrous materials based on absorbent fibers allowing the liquids to spread by lateral diffusion, which are commonly used in the field of hygiene products and dressings.

By way of example of such materials, mention may be made of materials based on absorbent fibers of plant origin, such as viscose, cellulose or derivatives thereof These materials may take the form of knits, wovens or nonwovens, obtained by a dry airlaid route or by a wet route, like papers.

The transfer structure may comprise a paper or a nonwoven, obtained by a dry airlaid route, based on absorbent fibers.

In the context of the present invention, nonwovens and papers are preferred. Among the nonwovens, preference is given to those based on absorbent fibers such as viscose or cellulose associated with non-absorbent fibers such as, for example, polyester or polyolefin fibers. By way of example of such nonwovens, mention may be made of the products marketed respectively by the companies Suominen Corp and Orsa under the trade names Fibrella® 2000 and Jettex® 1205 c or a hydrophilic nonwoven made up of 55% cellulose 45% polyester, of reference DR870, supplier Berkshire, and referred to hereinafter as a Berkshire nonwoven. Among papers, notable mention may be made of the paper referred to as the “Whatman” paper marketed by the Whatman company under the reference Benchote plus. The transfer structure may take the form of a drainage strip, directly in contact with a layer of an absorbent material of the receiving absorbent part, with no intermediate layer between them. As an alternative, an intermediate layer may be interposed, for example a perforated layer of a hydrophobic material, so as to reduce the risk of liquid returning toward the receiving absorbent part. As an alternative, the collection zone may be defined by a portion of the transfer structure directly in contact with the wound or with the interposition of a wound-contact layer interface dressing.

The transfer structure may have constituent elements which are closer together in the collection zone. These elements may lie in the same plane or conform to the relief of the region of the body or to the shape of the wound from which the physiological liquid to be collected originates.

Support

The article may comprise a support which may define at least part of the exterior surface of the article.

The support is preferably impermeable to water and external pathogenic microorganisms while at the same time being permeable to water vapor, so as both to avoid contact between the wound and external liquids and bacteria and to avoid maceration of the wound. The support is then termed “impermeable and breathable”.

The support is preferably thin and flexible, so as best to conform to the shape of the body and accompany the movements thereof without the risk of becoming detached. The support is advantageously conformable. Its thickness may be comprised between 100 and 600 μm, preferably between 250 and 500 μm.

The support may be made of a single material or an assembly of several materials.

The support may thus contain or consist of a film that is continuous and impermeable to liquids and bacteria but permeable to water vapor. By way of example of films that may be used, mention may be made of films made of polyetherurethane, of polyetheramide or polyetherester. The thickness of the film is, for example, comprised between 5 and 200 microns, preferably between 10 and 75 microns, more preferably still, between 10 and 50 microns.

The film advantageously has a moisture vapor transmission rate (MVTR) greater than 3 000 g/m²/24 hours, preferably greater than or equal to 7 000 g/m²/24 hours, more preferably still greater than or equal to 10 000 g/m²/24 hours. A technique for measuring the moisture vapor transmission rate in contact with liquid is described in standard NF-EN 13726-2 (Chapter 3.3).

Such films are commonly used in the creation of dressings and are for example made of polyurethane films, such as the films marketed by the company Exopack

Advanced Coating under the trade name INSPIRE.

The film may be replaced by a foam/film complex. The film may also be complexed with another material which then acts as a supporting framework to make the support more rigid.

Such a supporting framework may make it possible to make the support more rigid so that it does not curl up on itself after any potential peel-off protective films have been removed.

The supporting framework may be made of any perforated material such as a perforated film, a thermoplastic mesh, a textile such as a woven, a knit or a nonwoven for example, preferably an elastic one so that the article holds better on the skin.

When a perforated, film is used to make the supporting framework, the film is for example made of polyethylene or polypropylene. When a woven textile is used it is, for example, made of polyethylene terephthalate or of polyamide. The grammage of the supporting framework is preferably comprised between 10 and 500 g/m², for example between 20 and 300 g/m². Dressings with such supporting framework supports are described in application WO 2012/140377.

The film or complex acting as a support may be assembled with the other layers of the dressing using a discontinuous adhesive so as not to affect the water vapor permeability of the film or of the complex.

Another subject of the invention is the use of the article according to the invention to absorb a physiological liquid. Thus, in exemplary embodiments of the invention, this invention applies to the creation of disposable nappies and feminine sanitary products.

The invention may be better understood from reading the detailed description that follows, of some nonlimiting exemplary embodiments thereof and from studying the attached drawing in which:

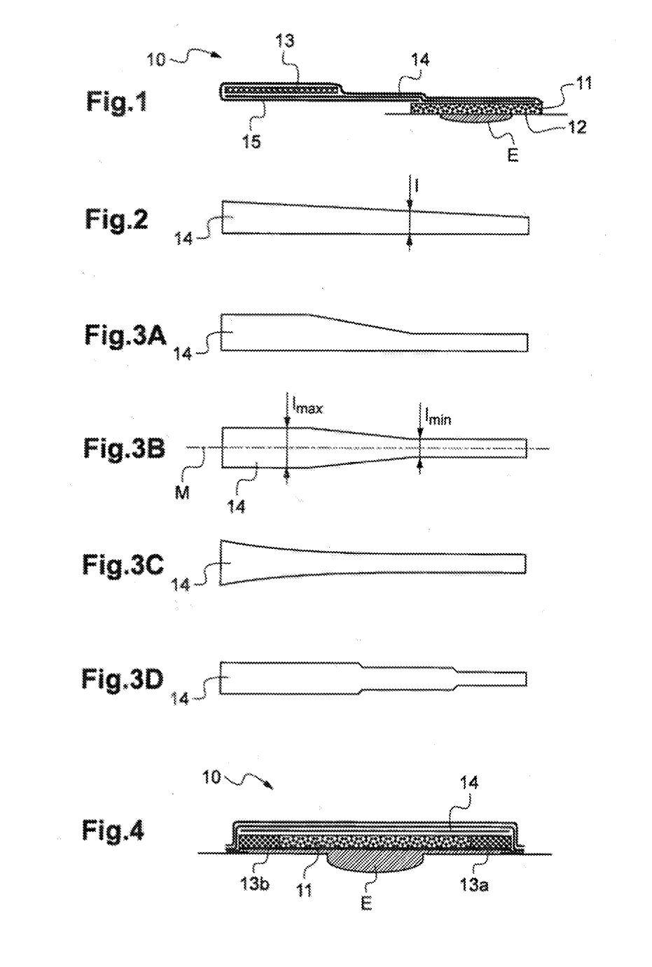

FIG. 1 schematically depicts, in cross section, one example of an article produced according to the invention,

FIG. 2 depicts in isolation, in a view from above, the transfer structure of the example of FIG. 1,

FIGS. 3A to 3D are views similar to FIG. 2 of alternative forms of the transfer structure,

FIG. 4 is a view similar to FIG. 1 of an alternative form of embodiment of an article,

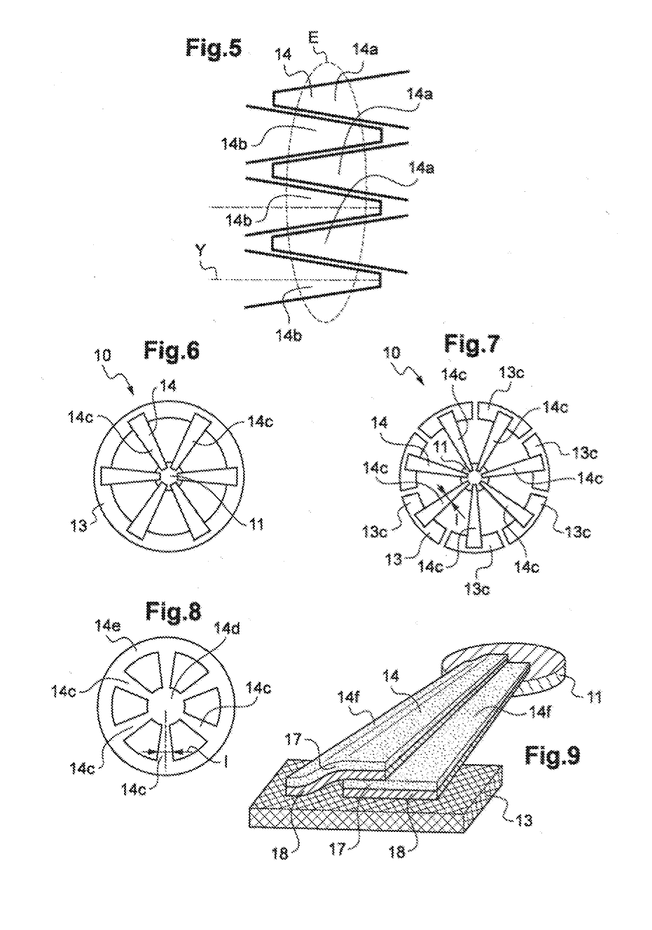

FIG. 5 illustrates the interpenetration of elements of an alternative form of embodiment of the transfer structure,

FIGS. 6 to 8 depict, in views from above, other alternative forms of transfer structures,

FIG. 9 is a schematic partial perspective view of another exemplary embodiment of the transfer structure,

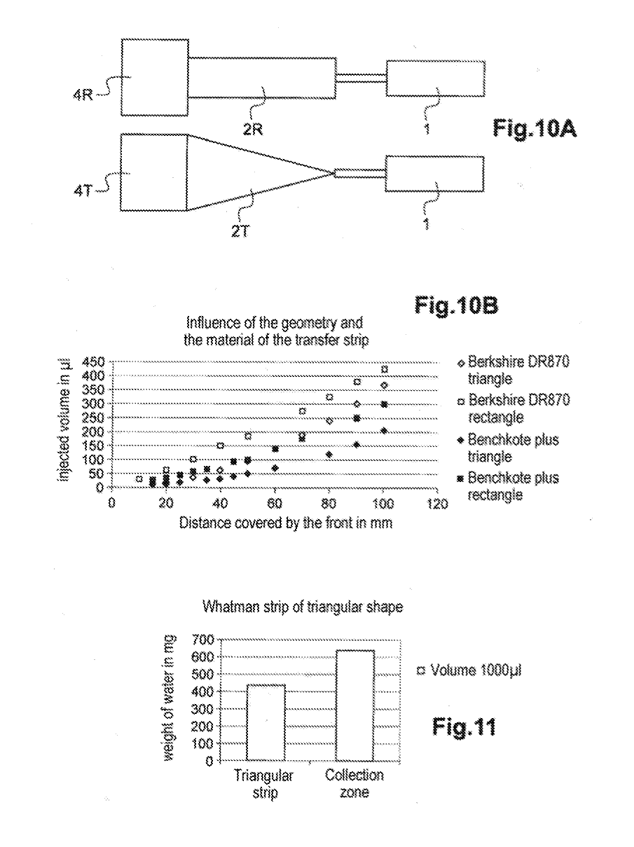

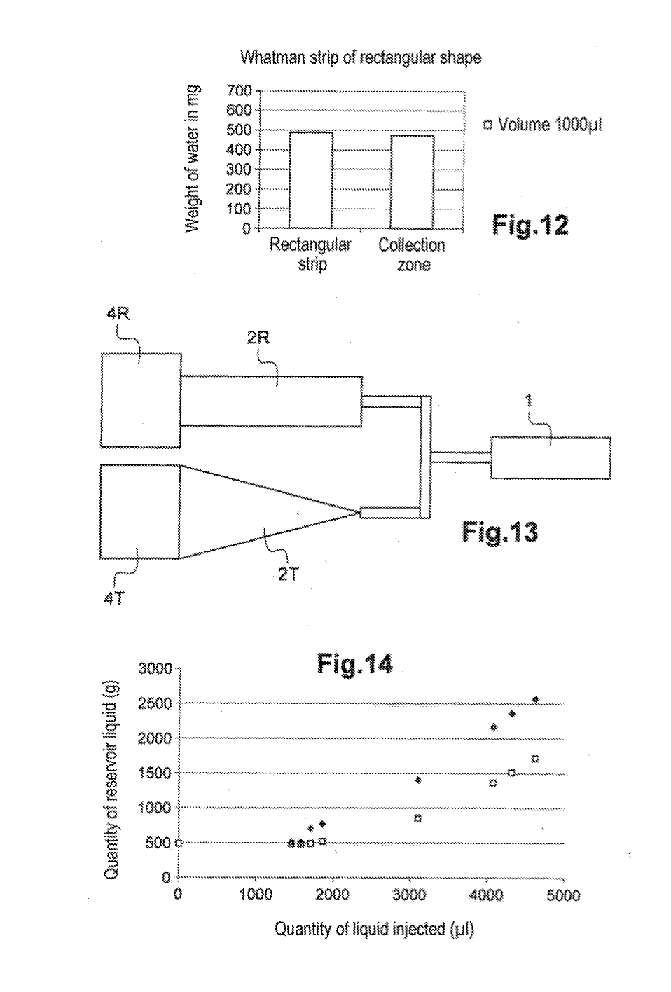

FIGS. 10A and 13 illustrate the methodologies used to carry out the comparative tests, and

FIGS. 10B, 11, 12 and 14 are graphs or tables illustrating experimental results.

The article 10 depicted in FIG. 1 is intended to be applied to part of a human or animal body, to absorb a physiological liquid such as an exudate E. It is preferably a dressing intended to be applied to a wound.

The article 10 may, as in the example illustrated, comprise a receiving absorbent part 11 intended to receive the exudate, defining an exudate collection zone 12, and an absorbent part forming the retention and/or evaporation zone 13 connected to the receiving absorbent part 11 by a transfer structure 14 according to the invention. In order to reduce the risk of exudate returning toward the receiving absorbent part 11, the article 10 may comprise an intermediate layer as defined hereinabove, not depicted in the drawing, situated between the transfer structure 14 and the receiving absorbent part 11.

The article 10 may also comprise a support 15 made of a layer impermeable to water, extending underneath the transfer structure 14 and which may also, as illustrated, cover the top of the part 13 forming the retention and/or evaporation zone and the transfer structure 14.

The receiving absorbent part 11, the transfer structure 14 and the support 15 are as defined above.

The transfer structure 14 has draining properties that allow liquid to be transferred by diffusion from the receiving absorbent part 11 toward the absorbent pan forming the retention and/or evaporation zone 13.

According to one aspect of the invention, the transfer structure 14 comprises absorbent fibers and offers for the transportation of the exudate a cross section that increases toward the part forming the retention and/or evaporation zone 13.

More particularly, the transfer structure 14 may When viewed from above, have a trapezoidal overall shape as illustrated in FIG. 2, of which the width l increases linearly with increasing proximity to the absorbent part forming the retention and/or evaporation zone 13. This increase in the width l makes it possible to increase the rate at which the liquid diffuses, and therefore the quantity of liquid collected by the absorbent part forming the retention and/or evaporation zone 13, thereby making it possible to improve the performance of the article in the collection of exudate.

The transfer structure 14 may have a thickness that is substantially constant over its entire length.

The invention is not restricted to a linear variation in the width l of the transfer structure 14 from one end to the other and this structure may, as illustrated in FIGS. 3A to 3D, for example have a width l that is constant over those portions of the transfer structure 14 that are superposed respectively with the receiving absorbent part 11 and with the absorbent part forming the retention and/or evaporation zone 13, as illustrated in FIG. 3A.

The variation in width l may be symmetric with respect to a midline M, as illustrated in FIG. 3B. The transfer structure 14 may have an overall shape that is symmetric with respect to said midline M.

The increase in width l may even be accomplished according to a more complex function, for example of nonlinear profile such as a hyperbolic, parabolic or some other profile, as illustrated in FIG. 3C, or even as step levels, as illustrated in FIG. 3D, with a constant width on each of the step levels.

The variation in width may be between values Iₘᵢₙ and Iₘₐₓ with, for example, a ratio Iₘₐₓ/Iₘᵢₙ greater than or equal to 50.

The thickness of the transfer structure 14 may vary, notably may increase in the direction of the absorbent part forming the retention and/or evaporation zone 13. However, the thickness is preferably constant, as this makes the article easier to manufacture.

The receiving absorbent pan 11 and the transfer structure 14 may be assembled using an adhesive which is, for example, applied discontinuously. The same goes for the assembling of the transfer structure 14 with the absorbent part forming the retention and/or evaporation zone 13.

The support 15 may extend over a short distance between the receiving absorbent part 11 and the transfer structure 14, The section of overlap is, for example, less than the surface area, when viewed from above, occupied by the receiving absorbent part, by a factor less than ½, or even less than ¼.

It is possible, as illustrated in FIGS. 1, 4, 6 and 7, 9 for the article 10 to comprise just one single part forming the retention and/or evaporation zone 13 which is offset with respect to the receiving absorbent part 11 so that when viewed from above there is no superposition between the part forming the retention and/or evaporation zone 13 and the receiving absorbent part 11, in these embodiments, the retention and/or evaporation zone 13 is positioned around the edge or at the periphery of the receiving absorbent zone 11, with no superposition between these two zones.

In the alternative form illustrated in FIG. 4, the article 10 comprises two opposite absorbent parts forming retention and/or evaporation zones 13a and 13b, which are positioned respectively on either side of the receiving absorbent part 11.

The transfer structure 14 may comprise several transfer elements 14a, 14b which interpenetrate above the receiving absorbent part 11, as illustrated in FIG. 5. The fact that each element 14a or 14b has a width that increases makes it easier for the elements to be imbricated and offers the possibility of having transfer elements which are very close together, although disjointed, at the receiving absorbent part 11.

The transfer elements 14a have, for example, mutually parallel longitudinal axes Y the same as the elements 14b.

The elements 14a, 14b, are, for example, cut in a single piece from a sheet of paper or nonwoven containing absorbent fibers as detailed above. The elements Ma may be joined together at one end, which is superposed with the absorbent part forming the retention and/or evaporation zone 13a. The same is true of the elements 14b.

The absorbent parts forming retention and/or evaporation zones 13a and 13b may be disjointed. As an alternative, they meet, and for example completely surround the receiving absorbent part 11.

The support 15 may externally cover the absorbent parts forming retention and/or evaporation zones 13a, 13b and the receiving absorbent part 11, except on the face thereof in contact with the wound.

The layout of the elements 14a, 14b may also be employed, within an article that has no receiving absorbent part 11 and/or no absorbent parts forming retention and/or evaporation zones 13a, 13b. In that case, the liquid collection zone is, for example, defined directly by the transfer structure 14. The transfer structure 14 may be used to evaporate the liquid with which it becomes laden, and the device may be created without an absorbent part forming the retention and/or evaporation zone in fluidic communication with the transfer structure.

FIG. 6 depicts an alternative form of embodiment in which the transfer structure 14 comprises radial transfer elements 14c each of which has a width I that increases from the collection zone 11, which is in the center, toward the absorbent part forming the retention and/or evaporation zone 13, which is peripheral and annularly continuous around the collection zone 11.

The elements 14c are, for example, arranged with even angular distribution about the collection zone 11.

The part forming the retention and/or evaporation zone 13 may be of angularly continuous annular shape, as illustrated in FIG. 6, or may be angularly discontinuous, as illustrated in FIG. 7, each element 14c of the transfer structure 14 then being able to communicate with a single respective reservoir-forming element 13c.

In the alternative form illustrated in FIG. 8, the elements 14c of the transfer structure 14 are produced as a single piece with a central part 14d which becomes superposed on any collection zone there might be and with a peripheral part 14e which is connected to any part there may be forming the retention and/or evaporation zone and/or which defines an evaporation zone.

The width l of each element 14c, measured at right angles to the corresponding radius, increases continuously from the central pan 1.4d toward the peripheral pan 14e.

In the examples that have just been described, comprising a transfer structure 14 comprising several elements connecting the collection zone to one or more parts forming retention and/or evaporation zones, these elements are disjointed and not superposed.

FIG. 9 illustrates the possibility of the transfer structure 14 comprising two elements 14f that are at least partially superposed. Each element 14f comprises a draining layer 18 which is covered on the top with a harrier layer 17 that is impermeable to water so that liquid diffusing, in the draining layer 18 may not reach the transfer element that covers it.

A superposition of the draining elements may make it possible to increase the throughput of the liquid diffusing in the transfer structure, for the same skin area occupied.

Comparative Tests

In order to demonstrate the fact that the shape of the transfer structure according to the invention makes it possible to increase the rate at which a liquid is drained from an inlet point toward an outlet point, comparative tests have been carried out and establish that a strip of triangular shape that is initially dry becomes moist more quickly than a strip of rectangular shape with the same surface area. The same is (quad when the strip is initially moist. The liquid is introduced at the narrower end in the case of the strip of triangular shape.

The methodology used to establish this was as follows. As described in FIG. 10A, the liquid is delivered to the strip (2R, 2T) that is to be tested by a syringe driver 1, as far as a part forming the retention and/or evaporation zone (4R, 4T). In this test, the retention zone (or reservoir) is made from the same material as the strip with which it is connected.

After a predefined volume has been delivered with the syringe driver, the liquid in the transfer strip (2R, 2T) and in the reservoir-forming part (4R, 4T) is measured by weighing.

The quantities of liquid present in strips of triangular and of rectangular shape and in the parts forming the retention and/or evaporation zones were able to be compared.

The injection rate is as high as 10 μL per minute. The strip tested is either rectangular (dimensions 45 mm * 210 mm) or triangular (base 90 mm-height 210 mm). Note that the two strips tested have the same thickness (thickness of around 100 μm), and therefore the same volume. The following are determined:
the propagation of the liquid front within the transfer strip as a function of the injected volume, this being determined before the liquid reaches the reservoir
after the liquid has reached the reservoir, the mass of the reservoir is determined, this being compared to the total mass injected.

FIG. 10B depicts the distance covered by the liquid front in the transfer strip, for two different materials: a Berkshire nonwoven (supplier Berkshire—reference BR870) and the Benchkote plus paper (supplier Whatman). These two materials are materials containing absorbent fibers. It may be seen, from studying FIG. 10, that for the same quantity of water injected, the distance covered by the liquid front (in millimeters) is greater in the case of the strip of triangular shape 21, that being true for both materials tested. That means that the liquid migrates more quickly through the strip of triangular shape 2T, particularly when the injected volume exceeds 40 μL.

That allows the liquid to reach the part thrilling the retention and/or evaporation zone 4T more quickly. The drainage capability of the triangular transfer strip 2T is greater than the drainage capability of the rectangular transfer strip 2R.

FIG. 11 depicts the volumes of liquid built up following injection of a 1 ml volume of liquid, for the case when the transfer strip and the reservoir are made from the Berkshire nonwoven. These volumes were estimated by cutting out the transfer strip and the collecting reservoir and weighing them. FIG. 12 shows similar results, obtained using the rectangular transfer strip made of Whatman paper. It is found that the collecting reservoir contained more liquid when the triangular transfer strip was used. Thus, when the strip is of triangular shape, the volume of liquid collected in the reservoir, at a given moment, is greater than that collected in the reservoir connected to a rectangular strip.

Another test was conducted, using a setup similar to that of the tests described previously, and which is depicted in FIG. 13. A transfer strip 2R, 2T is connected, at one of its ends, to a liquid injection device 1 and, at the opposite end, to a retention reservoir 4R, 4T made of an Airlaid material, described previously, marketed by the company Buckeye Steinfurt GmbH under the reference Vizorb® 3924. The transfer strips have dimensions identical to those described in the previous test. They are made of Berkshire nonwoven. For various injected quantities of liquid, the retention reservoir is weighed in order to determine the quantity of liquid drained by the transfer strip.

FIG. 14 depicts the quantity of liquid in the reservoir, expressed in grams, as a function of the quantity of liquid injected into the transfer strip, expressed in μl, for a triangular transfer strip and for a rectangular transfer strip. The transfer strips have dimensions similar to those described in the previous example. Their surface areas and thicknesses are the same, which means that the transfer strips have the same volume.

The initial mass of the reservoir 4R, 4T is 500 g. Beyond a certain volume of liquid injected, the liquid reaches the reservoir and the mass thereof increases. As a result, it is found that the liquid reaches the reservoir more rapidly when it is a triangular strip 2T that is used. That demonstrates that the triangular strip 2T has a superior draining effect to the rectangular strip 2R.

The invention may be applied to articles other than those intended to be applied to a wound, for example disposable nappies or feminine sanitary products.

1-16. (canceled)

17. An article having physiological-fluid absorption properties, comprising a liquid collection zone and a transfer structure , the latter comprising absorbent fibers and being designed to transport the liquid from said collection zone toward at least one retention and/or evaporation zone , said transfer structure having a cross section that increases in the direction of said retention and/or evaporation zone, over at least part of the path separating said collection zone from said retention and/or evaporation zone and being less absorbent on the one hand than the liquid collection zone and, on the other hand, than the retention and/or evaporation zone.

18. The article as claimed in claim 17, the increase in the cross section being obtained by increasing the width of the section.

19. The article as claimed in claim 18, the width increasing continuously over at least part of said path.

20. The article as claimed in claim 17, the ratio of the maximum width to the minimum width being greater than or equal to 1.5.

21. The article as claimed in claim 17, the thickness of the transfer structure being comprised between 50 and 1000 μm.

22. The article as claimed in claim 17, the transfer structure having a maximum absorption value of less than 500 g/m².

23. The article as claimed in claim 17, the transfer structure comprising at least two distinct elements for transporting the liquid toward the retention and/or evaporation zone.

24. The article as claimed in claim 23, said elements being arranged with an angular offset about a center of the article.

25. The article as claimed in claim 24, said elements being evenly angularly distributed.

26. The article as claimed in claim 23, said elements extending along substantially mutually parallel axes.

27. The article as claimed in claim 17, the transfer structure comprising elements draining the liquid in different directions.

28. The article as claimed in claim 27, the elements draining the liquid toward at least two parts that form respective different retention and/or evaporation zones.

29. The article as claimed in claim 27, said elements interpenetrating one another at the collection zone.

30. The article as claimed in claim 27, the elements being produced as a single piece by being cut from a material in sheet form.

31. The article as claimed in claim 30, the elements being joined together at the collection zone.

32. The article as claimed in claim 30, the elements being joined together at the retention and/or evaporation zone.

33. The article as claimed in claim 17, the collection zone being defined by a receiving absorbent part containing a cellular material.

34. The article as claimed in claim 17, the transfer structure comprising a nonwoven, obtained by a dry airlaid route, based on absorbent fibers or a paper.
